(19) United States
(12) Patent Application Publication
(10) Pub. No.: US 2013/0139819 A1
(43) Pub. Date: Jun. 6, 2013

(54) SLEEP APNEA TREATMENT DEVICE

(71) Applicants: Ronald D. Chervin, Ann Arbor, MI (US); Katya B. Christenson, Sioux Falls, SD (US); Steven M. Fannon, Novi, MI (US); Joseph W. Jacquemin, Panama City Beach, FL (US); Davina A. Widjaja, Jakarta Utara (ID); Kathleen Sienko, Ann Arbor, MI (US)

(72) Inventors: Ronald D. Chervin, Ann Arbor, MI (US); Katya B. Christenson, Sioux Falls, SD (US); Steven M. Fannon, Novi, MI (US); Joseph W. Jacquemin, Panama City Beach, FL (US); Davina A. Widjaja, Jakarta Utara (ID); Kathleen Sienko, Ann Arbor, MI (US)

(73) Assignee: THE REGENTS OF THE UNIVERSITY OF MICHIGAN, Ann Arbor, MI (US)

(21) Appl. No.: 13/514,616

(22) PCT Filed: Dec. 10, 2010
(86) PCT No.: PCT/US2010/059881

§ 371 Date: Jan. 18, 2013

Related U.S. Application Data
(60) Provisional application No. 61/285,318, filed on Dec. 10, 2009.
(60) Provisional application No. 61/323,736, filed on Apr. 13, 2010.

(51) Int. Cl. A61M 16/00; A61M 16/08; A61M 16/06; A61M 16/20

(52) U.S. Cl. CPC A61M 16/0075; A61M 16/20; A61M 16/0875; A61M 16/06
USPC 128/205.16

(57) ABSTRACT

A sleep apnea treatment device that includes a first chamber, a second chamber, and an energy storage component. The first chamber receives a patient's exhaled breath, which causes the second chamber to be expanded with fresh air that is drawn in during expansion of the second chamber. Energy from the patient's exhaled breath is stored in the energy storage component and then used to expel the air in the second chamber under positive pressure into the patient's airway. The first chamber can expand during the patient's exhaled breath which causes a plate or other movable member to simultaneously expand the second chamber to thereby draw in the fresh air that is subsequently expelled to the patient using the energy in the energy storage component.

### TECHNICAL FIELD

This invention relates generally to treating obstructive sleep apnea (OSA) disorders, and more particularly to devices used to treat OSA.

### BACKGROUND

Obstructive sleep apnea (OSA) is a common human sleep disorder in which throat muscles relax during sleep and narrow (hypopnea) or altogether close (apnea) the upper airway. When this happens, breathing is ceased temporarily and the brain is aroused to open the airway. Constant arousals and drops in oxygen levels disrupt sleep and can lead to cognitive, cardiovascular, and metabolic morbidity, and in some cases can contribute to daytime sleepiness, heart troubles, hypertension, arrhythmia, myocardial infarction, stroke, diabetes, metabolic syndrome, and a shortened lifespan, among other concerns.

Continuous positive airway pressure (CPAP) machines have been developed to treat OSA. The CPAP machines are used by a patient while sleeping, and work by splinting the upper airway open under positive pressure to permit continued breathing during sleep. The machines commonly include an airflow generator, a hose connected to the generator at one end, and a mask connected to the hose at the other end of the hose. The airflow generator is usually a fan or other blower which requires electrical power from an electrical outlet or on rare occasions battery, restricting their use accordingly.

### SUMMARY

According to one embodiment, a positive airway pressure device includes an energy accumulator and an air delivery subsystem. The energy accumulator includes a first port that receives exhaled breath from a patient and includes one or more components that store energy from the received breath. The air delivery subsystem has a second port and is coupled to the energy accumulator. The air delivery subsystem generates a pressurized volume of fresh air by using the stored energy, and the subsystem provides the pressurized volume of fresh air to the second port for eventual delivery to the patient.

According to another embodiment, a sleep apnea treatment device includes a first chamber, a second chamber, and an energy storage component. The first chamber receives exhaled breath from a patient and is inflated by the exhaled breath. The second chamber expands its size in response to the inflation of the first chamber, and upon expansion draws in fresh air that is eventually inhaled by the patient. The energy storage component interacts with the second chamber. When the patient terminates exhaling and initiates inhaling, the energy storage component facilitates contraction of the second chamber and the previously drawn-in fresh air is expelled under positive pressure out of the second chamber for delivery to the patient.

According to yet another embodiment, a sleep apnea treatment device includes a housing, a first expandable and contractible chamber, a second expandable and contractible chamber, and an energy storage component. The first chamber is defined in part or more by a stationary plate, a movable plate, and a first outer wall connected between the plates. The second chamber is defined in part or more by the stationary plate, the movable plate, and a second outer wall connected between the plates. The energy storage component interacts with the movable plate to bias the movable plate to a position in which the first and second chambers are contracted in size. When the patient exhales, the first chamber receives the exhaled breath and is inflated thereby and expands its size. Also, the movable plate causes the second chamber to expand its size and the second chamber draws-in fresh air when it expands. When the patient then subsequently inhales, the energy storage component facilitates movement of the movable plate in order to contract the size of the first and second chambers. When the chambers are contracted, the exhaled breath leaves the first chamber to the atmosphere, and the fresh air is expelled under positive pressure out of the second chamber for delivery to the patient.

Also provided in accordance with an embodiment of the invention is a method of treating sleep apnea. The method comprises the steps of storing energy received from exhalation of air by a patient, creating a pressurized volume of fresh air using the stored energy, and delivering the fresh air to the patient during inspiration.

### BRIEF DESCRIPTION OF THE DRAWINGS

Preferred exemplary embodiments of the invention will hereinafter be described in conjunction with the appended drawings, wherein like designations denote like elements, and wherein:

FIG. 1 is a perspective view of a first embodiment of a sleep apnea treatment device;

FIG. 2 is a side view of the sleep apnea treatment device of FIG. 1 shown in an inflated state, and shown with outer walls taken away to expose internal components of the device;

FIG. 3 is a side view of the sleep apnea treatment device of FIG. 1 shown in a deflated state, and shown with outer walls taken away to expose internal components of the device;

FIG. 4 is a partially exploded view of a part of the sleep apnea treatment device of FIG. 1;

FIG. 5 is a partially exploded view of a part of the sleep apnea treatment device of FIG. 1;

FIG. 6 is a top view of an embodiment of a plate that is used with the sleep apnea treatment device of FIG. 1;

FIG. 7 is a perspective view of an embodiment of an exhalation assembly of the sleep apnea treatment device of FIG. 1;

FIG. 8 is a perspective view of an embodiment of an inhalation assembly of the sleep apnea treatment device of FIG. 1;

FIG. 9 is an exploded view of the inhalation assembly of FIG. 8;

FIG. 10 is a perspective view of an embodiment of a valve assembly;

FIG. 11 is a perspective view of the sleep apnea treatment device of FIG. 1, showing an embodiment of a hose assembly that can be used therewith;

FIG. 12 is a perspective view of a second embodiment of a sleep apnea treatment device;

FIG. 13 is a partially exploded view of the sleep apnea treatment device of FIG. 12;

FIG. 14 is a diagrammatic view of a third embodiment of a sleep apnea treatment device;

FIG. 15 is another diagrammatic view of the sleep apnea treatment device of FIG. 14;

FIG. 16 is an x-y graph with the exhalation pressure required (centimeters of H₂O) on the y-axis, and the weight applied to test exhalation pressure (grams) on the x-axis;

FIG. 17 is an x-y graph with the inhalation pressure measured (centimeters of H₂O) on the y-axis, and the weight applied to test inhalation pressure (grams) on the x-axis;

FIG. 18 is a simple spring, mass, and dampener system model of the sleep apnea treatment device of FIG. 1;

FIG. 19 is a perspective view of a fourth embodiment of a sleep apnea treatment device;

FIG. 20 is a front view of the sleep apnea treatment device of FIG. 19;

FIG. 21 is a cross-sectional view taken at 21-21 in FIG. 20;

FIG. 22 is a cross-sectional view taken at 22-22 in FIG. 20;

FIG. 23 is a front view of an embodiment of a valve assembly that can be used with the sleep apnea treatment device of FIG. 19; and

FIG. 24 is a front view of an embodiment of a valve that can be used in the sleep apnea treatment device of FIG. 19.

### DETAILED DESCRIPTION OF THE PREFERRED EMBODIMENTS

The drawings show several embodiments of a sleep apnea treatment device, also called a positive airway pressure device, that is used to alleviate obstructive sleep apnea (OSA) disorders including hypopnea in which the upper airway of a patient is narrowed, and apnea in which the upper airway is closed. The device communicates with the patient's upper airway and works by splinting the airway open under positive pressure to allow continued breathing during sleep. The energy required for operating the sleep apnea treatment device can be derived solely from the patient, although electrical sources of backup or assistive power can be used if desired. In the illustrative embodiments, energy from the patient's exhaled breath is utilized to draw-in the fresh air that is eventually expelled to the patient at a pressure above atmospheric pressure.

This can be done by way of a passive mechanical operating system where movement is initiated from the patient's exhaled breath and is not assisted by an external power source, such as an electrical outlet or a battery as is the case in an active operating system like conventional continuous positive airway pressure (CPAP) machines. This does not necessarily mean that electrical devices are not used in or associated with the disclosed sleep apnea treatment devices, such as electrical devices which can convert mechanical input to electrical or magnetic output and which could serve to supplement or amplify the energy generated via the patient's exhaled breath. It merely means that the patient's exhaled breath constitutes the primary, if not total, source of energy used in operating the system. As compared to the conventional CPAP machines, the sleep apnea treatment device can be implemented as a passive CPAP machine that is a smaller, lighter, quieter, less expensive, and more portable piece of equipment, and thus is better suited for use in lesser developed parts of the world where electricity is unavailable or unreliable.

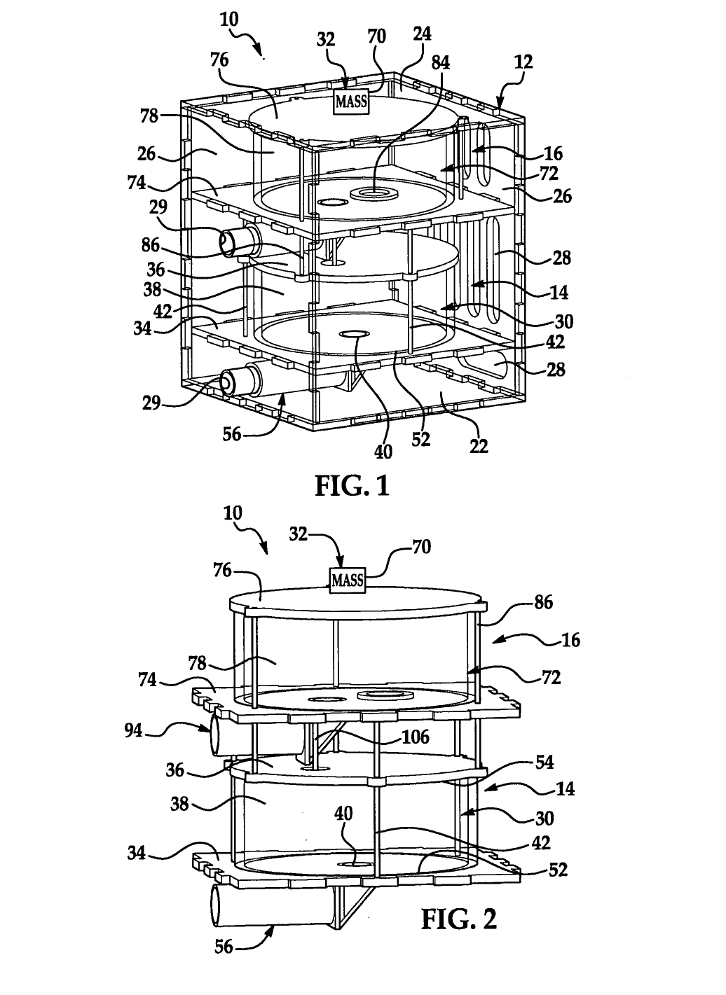

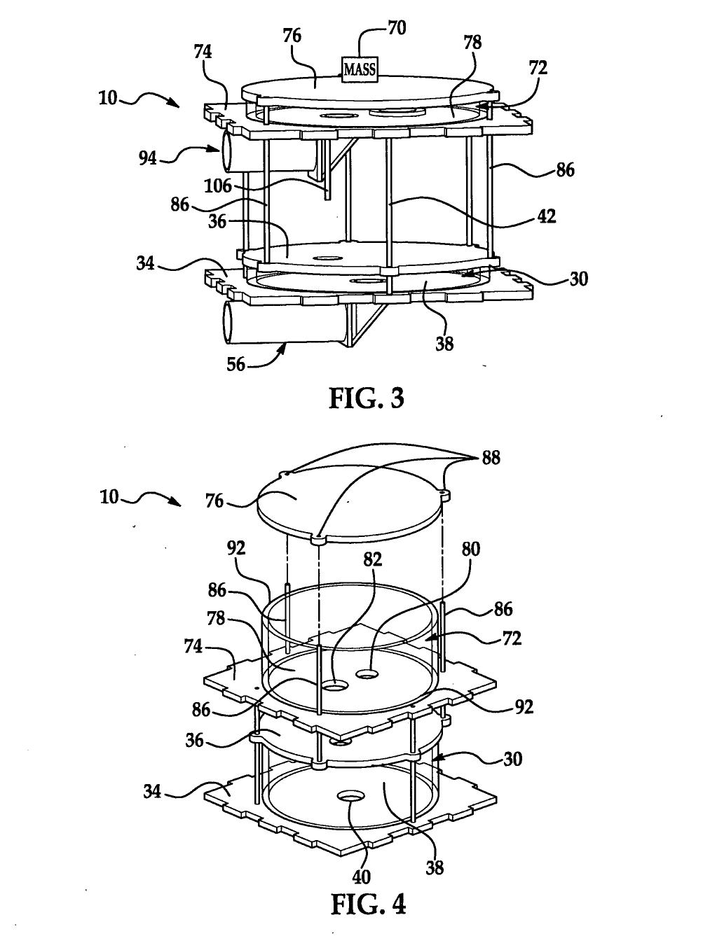

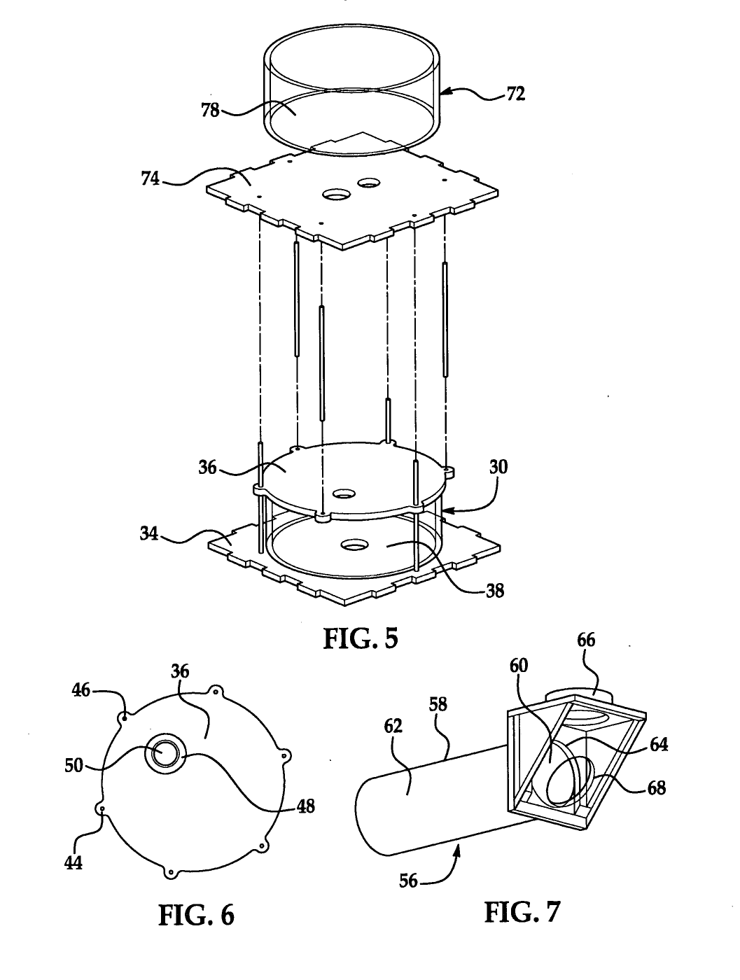

FIGS. 1-11 depict a first embodiment of a sleep apnea treatment device 10 that includes a housing 12, an energy accumulator 14, an air delivery subsystem 16, a hose assembly 18 to and from the device, and a mask 20 for a patient.

The housing 12 provides a structure for containing and supporting some of the other components of the sleep apnea treatment device 10. In general, the housing 12 is designed and constructed to be, among other things, durable, compact, and sturdy. The housing 12 can be made of a PVC material, or another hard plastic or material, and can be manufactured by way of an injection molding process, or another suitable process. Referring to FIG. 1, the housing 12 generally includes a base 22 to stabilize the device 10, a top wall 24, and multiple side walls 26. The base 22 and walls have interconnecting and complementary male and female structures around their respective peripheries for joining the separate pieces together; though shown as separate and distinct pieces, one or more of the base and walls could make-up a one-piece construction. One of the side walls 26 can have numerous vent openings 28 located therein to allow gases to flow in and out of the housing 12, and another one of the sides walls can have a pair of openings 29 for connections between the housing and the hose assembly 18.

The energy accumulator 14 receives the patient's exhaled breath and captures and temporarily stores energy derivable therefrom. The energy accumulator 14 can take many forms with different constructions, arrangements, and energy capturing and storage capabilities and operations including by way of mechanical movement and electrical conversion. The first embodiment of the energy accumulator 14 operates by way of mechanical movement, and generally includes a first chamber 30 and an energy storage component 32.

The first, or exhalation, chamber 30 confines a volume and inflates when receiving the patient's exhaled breath and deflates when expelling the used air to the atmosphere. The size of the first chamber 30 varies and expands and contracts during use of the sleep apnea treatment device 10. One example maximum inflatable volume of the first chamber 30 is 650 ml, which is based on a slightly above average volume of a human's exhaled breath during sleep; of course, the exact maximum volume can be greater or less than this and will be dictated mostly by the expected volume of the particular patient's exhaled breath. Referring to FIGS. 1-5, the first chamber 30 is located inside of the housing's structure and at the base 22 near a vertical bottom of the device 10. The first chamber 30 is defined in part by a first plate 34, a second plate 36, and an outer wall 38, which are joined together in an air-tight manner. The first, or stationary, plate 34 is connected to the side walls 26 and does not move during use of the sleep apnea treatment device 10. It has a first opening or port 40 for the ingress and egress of exhaled breath in and out of the first chamber 30 during use of the device 10. Like pieces of the housing 12, the first plate 34 can be made of a PVC material, or another hard plastic or material, and can be manufactured by way of an injection molding process, or another suitable process.

The second, or movable, plate 36 is slidably connected to the first plate 34 by way of multiple rods 42, and during use of the device 10 reciprocates linearly up and down to expand and contract the size of the first chamber 30. The rods 42 guide the reciprocation of the second plate 36, and are fixed at one end to the first plate 34 and at their other end to another plate of the device 10. The rods 42 can be made of aluminum, steel, or another suitable rigid material such as a hard plastic, and could have a ground or polished outer surface to minimize friction with the second plate 36. The second plate 36, on the other hand, can be made of a PVC material or another hard plastic or material, and can be manufactured by way of an injection molding process, or another suitable process; of course, the second plate could also be made of aluminum, steel, or another suitable metal material. Referring to FIG. 6, the second plate 36 has a generally disc shape, has three guide holes 44 located around its periphery for being carried by the rods 42, and has three holes 46 with female threads for fastening with other rods of the device 10. The second plate 36 also has a second opening 48 for fitting a relief valve 50 therein. The relief valve 50 is used to accommodate volumes of exhaled breath which exceed the maximum inflatable volume of the first chamber 30. The relief valve 50 includes a one-way downward opening rubber flapper which, as shown best in FIG. 2, abuts a cam 106 to open and permit exit of exhaled breath through the relief valve when the chamber 30 is fully inflated. The exiting exhaled breath thereafter no longer causes significant further upward movement of the second plate 36.

The outer wall 38 extends and is interconnected between the first and second plates 34, 36, forms seals therewith, and allows the first chamber 30 to expand and contract in size. The outer wall 38 is made of a flexible material in the sense that it can be foldable, pliable, or otherwise capable of reciprocal collapsing and extending as breath is inhaled and exhaled, respectively. For example, the outer wall 38 can be made out of a bag material, a vinyl material, a PVDC material, can have a bellows-type construction, can be a flexible material supported in part by a helical wire, or can be another material and construction. Referring to FIGS. 1-5, the outer wall 38 has a first and second open end 52, 54 that are respectively attached to the first and second plate 34, 36 such as by adhesive, stitching, stapling, or another way. The outer wall 38 could also be attached to the plates 34, 36 in a way that allows removal of the outer wall for periodic cleaning by the patient. For example, the outer wall 38 and the plates 34, 36 could be attached via a male and female turn-and-lock structure, where a ring structure would be located at the open ends 52, 54 and an annular recessed structure would be located on the plates 34, 36. In another embodiment, the first chamber 30 can be defined primarily by a flexible wall like the outer wall 38 in the form of a bladder, for example, which is squeezed and/or stretched and collapsed and/or relaxed between the first and second plates 34, 36 during expansion and contraction.

Referring to FIGS. 2, 3, and 7, a first, or exhalation, valve assembly 56 communicates with the first chamber 30 and regulates the ingress and egress of exhaled breath into and out of the first chamber. The first valve assembly 56 is attached to a bottom side of the first plate 34 at the first opening 40. In the embodiment shown, the first valve assembly 56 includes a body 58 and a flapper 60. The body 58 can be made of a PVC material or another hard plastic or material, and can be manufactured by way of an injection molding process or another suitable process. The body 58 has an inlet tube 62 for connection to the hose assembly 18, an inlet port 64 that is opened and closed by the flapper 60, a chamber port 66 that communicates directly with the first chamber 30, and an outlet port 68 that communicates directly with the inside of the housing's structure and indirectly with the atmosphere via openings 28.

Referring particularly to FIG. 7, the flapper 60 is made of a rubber material and opens one-way in a direction away from the inlet tube 62 and otherwise rests in a position where it closes the inlet port 64. In use, exhaled breath enters the valve assembly 56 through the inlet tube 62 and causes the flapper 60 to pivot exposing and opening the inlet port 64. When pivoted, the flapper 60 plugs the outlet port 68 so that the incoming exhaled breath does not exit the outlet port and instead passes through the chamber port 66 and into the first chamber 30. Conversely, when the first chamber 30 contracts the flapper 60 plugs the inlet port 64 and exiting exhaled breath passes through the chamber port 66 and through the outlet port 68. In other embodiments, the first valve assembly could be an off-the-shelf component purchased from a supplier and could have another construction.

The energy storage component 32 temporarily stores energy resulting from the patient's exhaled breath. The energy storage component 32 can take many forms with different constructions, arrangements, and energy storage capabilities and operations. Referring to FIGS. 1-3, this embodiment of the energy storage component 32 is a biasing member such as a mass 70. The mass 70 is coupled to the first chamber 30 and resists expansion of the first chamber and upward movement of the second plate 36, and promotes contraction of the first chamber and downward movement of the second plate. The mass 70 acts directly or indirectly on the first chamber 30 and, in association with gravity, exerts a weight or resistance force against upward movement of the second plate 36. In general, increasing potential energy is produced at the mass 70 as the mass is displaced a vertical distance upward upon expansion of the first chamber 30.

The mass 70 is a separate and distinct component that is placed or fitted on top of another movable plate 76 (discussed below) which itself is connected to the second plate 36 by way of rods. The exact value of the mass 70 will be influenced by, among other factors, the combined mass of the other movable components (e.g., plates 36, 76, etc.), the resulting pressure provided in the first chamber 30 by the patient's exhaled breath, and the desired pressure of the expelled fresh air. In other embodiments, the mass need not be a separate component, but can be provided by selecting a suitable mass for one or both plates, 36, 76 where the plates themselves provide the function of stored energy; for example, by suitable selection of materials and thickness. In other embodiments, the mass could be a container or pouch that is filled with water, sand, or another material to provide an adjustable weight; in this example, the container could be indexed to indicate the corresponding weight according to the amount of material filled or taken out. In yet another example, the mass as a separate component could also be located on the second plate 36. By providing an adjustable mass, any of a wide range of pressures can be generated that might be required by a particular patient.

The air delivery subsystem 16 interacts with the energy accumulator 14 and generates a positively pressurized volume of fresh air in cooperation with the stored energy of the energy accumulator, and delivers the fresh air to the hose assembly 18. The air delivery subsystem 16 can take many forms with different constructions, arrangements, and pressure generating capabilities and operations including by way of mechanical movement. The first embodiment of the air delivery subsystem 16 includes a second chamber 72 to draw-in fresh air.

The second, or inhalation, chamber 72 confines a volume and inflates as it draws-in fresh air from outside of the housing 12, and deflates to expel air to the hose assembly 18 and eventually to the patient. The size of the second chamber 72 varies and expands and contracts during use of the sleep apnea treatment device 10. Like the first chamber 30, one example maximum inflatable volume of the second chamber 72 is 650 ml; of course, the exact maximum volume can be greater or less than this and can be dictated by the expected volume of the particular patient's exhaled breath, the desired volume of the particular patient's inhaled breath, or both. Referring to FIGS. 1-5, the second chamber 72 is located inside of the housing 12 and vertically above the first chamber 30 to provide a stacked top-and-bottom chamber arrangement. The second chamber 72 is defined in part by a third plate 74, a fourth plate 76, and an outer wall 78, which are joined together in an air-tight manner.

The third, or stationary, plate 74 is connected to the side walls 26 and does not move during use of the sleep apnea treatment device 10. It has a third and fourth opening 80, 82 for the ingress and egress of fresh air during use. Like pieces of the housing 12, the third plate 74 can be made of a PVC material, or another hard plastic or material, and can be manufactured by way of an injection molding process, or another suitable process. A flapper 84 is located in the third opening 80 and is made of a rubber material. The flapper 84 rests in a closed position and opens one-way in a vertical direction to permit the entrance of fresh air into the second chamber 72 upon expansion of the second chamber. The flapper 84 also operates as a safety valve-that opens to let fresh air into the second chamber 72 if the patient inhales an unusually large or irregular breath that exhausts the remaining capacity of the pressurized second chamber.

Referring to FIGS. 1-4, the fourth, or movable, plate 76 is fixed to the second plate 36 by way of multiple rods 86, and reciprocates linearly up and down in unison and simultaneously with the second plate to expand and contract the size of the second chamber 72. As the second chamber 72 expands, a partial vacuum is created inside of the second chamber which draws fresh air into the second chamber through the third opening 80. The rods 86 are fitted with male threads on each of their ends, and are screwed into the fourth plate 76 and the second plate 36. The rods 86 can be made of aluminum, steel, or another suitable rigid material such as a hard plastic. The fourth plate 76 can be made of a PVC material, another hard plastic, or a metal material, and can be manufactured by way of an injection molding process, or another suitable process. Like the second plate 36, the fourth plate 76 has a generally disc shape, and has three holes 88 located around its periphery that are fitted with female threads for fastening with the male threads of the rods 86.

The outer wall 78 extends and is interconnected between the third and fourth plates 74, 76, forms seals therewith, and allows the second chamber 72 to expand and contract in size. The outer wall 78 is made of a flexible material which can, but need not be, the same material as used for the outer wall 38 of the first chamber 30. For example, the outer wall 78 can be made out of a bag material, a vinyl material, a PVDC material, can have a bellows-type construction, can be a flexible material supported in part by a helical wire, or can be another material and construction. Referring to FIGS. 1-5, the outer wall 78 has a first and second open end 90, 92 that are respectively attached to the third and fourth plate 74, 76 such as by adhesive. The outer wall 78 could be attached to the plates 74, 76 in other ways that allow removal of the outer wall for periodic cleaning by the patient. For example, the outer wall 78 and the plates 74, 76 could be attached via a male and female turn-and-lock structure, where a ring structure would be located at the open ends 90, 92 and an annular recessed structure would be located on the plates 74, 76. In another embodiment, the second chamber 72 is defined primarily by a flexible wall like the outer wall 78 in the form of a bladder, for example, which is squeezed and stretched between the third and fourth plates 74, 76 for expansion and contraction.

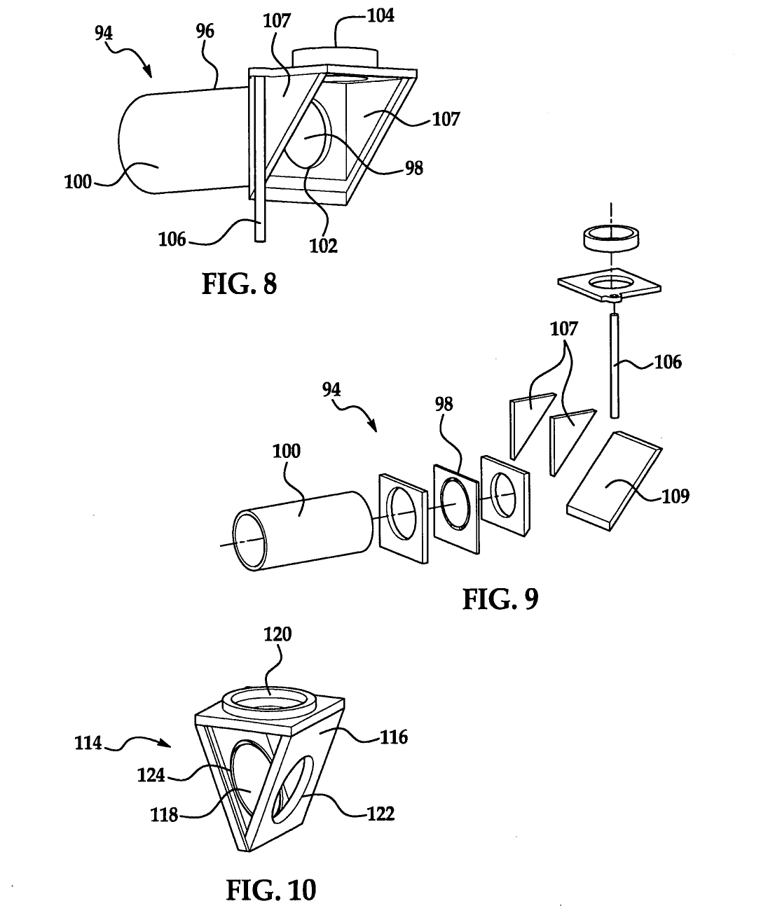

Referring to FIGS. 2, 3, 8, and 9, a second, or inhalation, valve assembly 94 communicates with the second chamber 72 and regulates the egress of fresh air out of the second chamber. The second valve assembly 94 is attached to a bottom side of the third plate 74 at the fourth opening 82. In this embodiment, the second valve assembly 94 includes a body 96 and a flapper 98. The body 96 can be made of a PVC material or another hard plastic or material, and can be manufactured by way of an injection molding process, or another suitable process. The body 96 has an outlet tube 100 for connection to the hose assembly 18, an inlet port 102 that is opened and closed by the flapper 98, a chamber port 104 that communicates directly with the second chamber 72, and a cam 106 that interacts with the relief valve 50. The body 96 also has a pair of side walls 107, and a bottom wall 109 (bottom wall removed in FIG. 8 to show internal components). The flapper 98 is made of a rubber material and opens one-way in a direction toward the outlet tube 100, and otherwise rests in a closed position. In use, expelled fresh air enters the second valve assembly 94 through the chamber port 104 and causes the flapper 98 to pivot exposing and opening the inlet port 102. The expelled fresh air then travels through the outlet tube 100 and to the hose assembly 18. In other embodiments, the second valve assembly could be an off-the-shelf component purchased from a supplier and could have another construction.

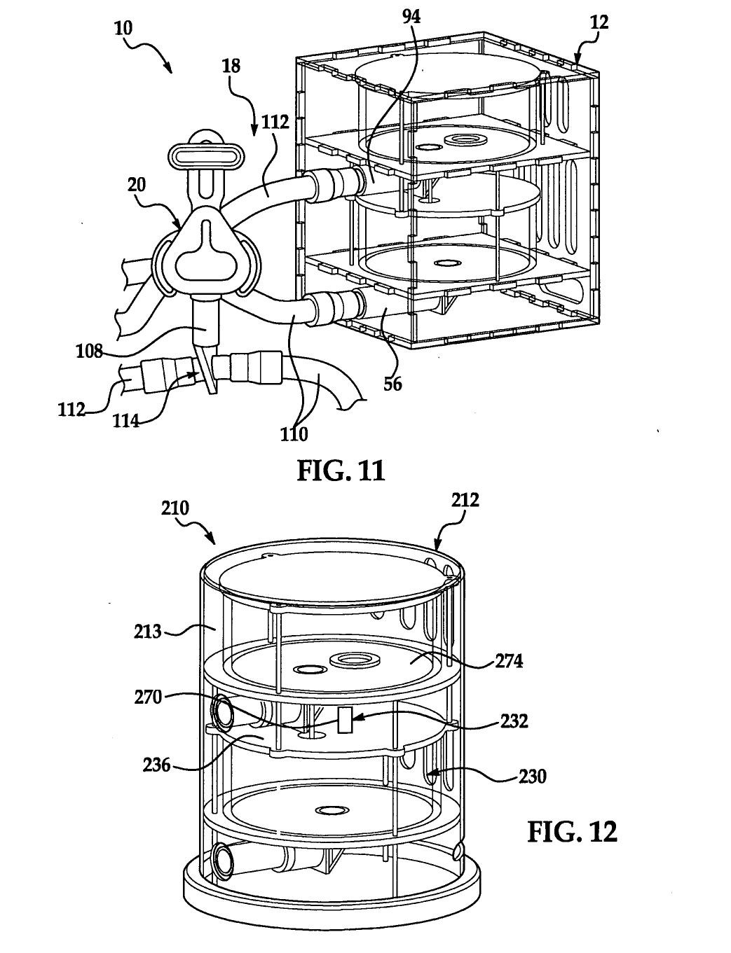

The hose assembly 18 communicates the first and second chambers 30, 72 with the mask 20, and carries and delivers exhaled breath to the first chamber and fresh air from the second chamber. Referring to FIG. 11, the hose assembly 18 includes a first hose 108 fitted directly to the mask 20, a second hose 110 fitted directly to the first valve assembly 56, and a third hose 112 fitted directly to the second valve assembly 94. Referring to FIG. 10, the hose assembly 18 also includes a valve assembly 114. The valve assembly 114 is located at an intersection of the first, second, and third hoses 108, 110, 112, as shown in FIG. 11. The valve assembly 114 regulates gas flow from the patient to the first chamber 30 and from the second chamber 72 to the patient. The valve assembly 114 permits the patient's exhaled breath to flow from the first hose 108 and to the second hose 110, permits the fresh air to flow from the third hose 112 and to the first hose, prevents the patient's exhaled breath from flowing from the first hose and to the third hose, and prevents the fresh air from flowing from the third hose and to the second hose.

In this embodiment, the valve assembly 114 includes a body 116 and a flapper 118. The body 116 can be made of a PVC material or another hard plastic or material, and can be manufactured by way of an injection molding process, or another suitable process. The body 116 has a first port 120 that communicates with the first hose 108, a second port 122 that communicates with the second hose 110, and a third port 124 that communicates with the third hose 112. The flapper 118 is made of a rubber material and opens one-way in the direction of the second port 122, and otherwise rests in a position where it plugs and closes the third port 124. In use, fresh air flowing from the third hose 112 causes the flapper 118 to pivot and open the third port 124. When pivoted, the flapper 118 plugs and closes the second port 122. In other embodiments, the valve assembly could be an off-the-shelf component purchased from a supplier and could have another construction. And in other embodiments, the hose assembly 18 could be a single hose, but more dead space might exist in a single hose as compared to the hose assembly of FIG. 11.

Referring to FIG. 11, the mask 20 is worn by the patient and could be a nose mask or a full nose and mouth mask; however, the design used should be able to capture a substantial amount of the exhaled breath under pressure for use in storing enough energy to provide a positive flow of fresh air back to the patient. Numerous types of masks can be used, including commercially available masks such as the ComfortGel Nasal Mask sold by Royal Philips Electronics, globally headquartered at Amstelplein 2, Breitner Center, P.O. Box 77900, 1070 MX Amsterdam, The Netherlands, (www.usa.philips.com). Another suitable mask is called the Mirage Swift II sold by ResMed Corp., located at 9001 Spectrum Center Blvd., San Diego, Calif. 92123, (www.resmed.com). Most masks, including the ComfortGel Nasal Mask, have one or more vents or ports for exiting exhaled breath. For use with the sleep apnea treatment device 10, these vents or ports can be plugged so that exhaled breath does not exit the vents or ports and instead flows through the hose assembly 18.

The general movement and operation of the sleep apnea treatment device 10 can be described in terms of physics. The patient's exhaled breath exerts a force against the movable plate 36 of the first chamber 30, which produces a pressure on the plate and causes the plate to move vertically upward against the force of gravity acting on the combined mass of the movable elements (mass 70, plates 36, 76, rods 86, etc.). The exact vertical distance of the movable plate 36 will depend on, among other things, the volume of the patient's exhaled breath and the volume of the first chamber 30. The movable plate 76 of the second chamber 72 moves in unison with the movable plate 36 of the first chamber 30 which vertically displaces the weight (i.e., the mass 70) and generates potential energy in the weight and in the plates 36, 76. After exhalation and at the beginning of inhalation, the stored potential energy converts to kinetic energy and causes the mass 70 and the plates 36, 76 to fall toward its resting position where the first and second chambers 30, 72 are deflated. The falling movable plate 76 pressurizes the fresh air (i.e., greater than atmospheric pressure) in the second chamber 72 and expels it out of the second chamber. The pressure of inhalation is similar to the pressure caused by exhalation, though some energy can be lost in the sleep apnea treatment device 10 such as through friction.

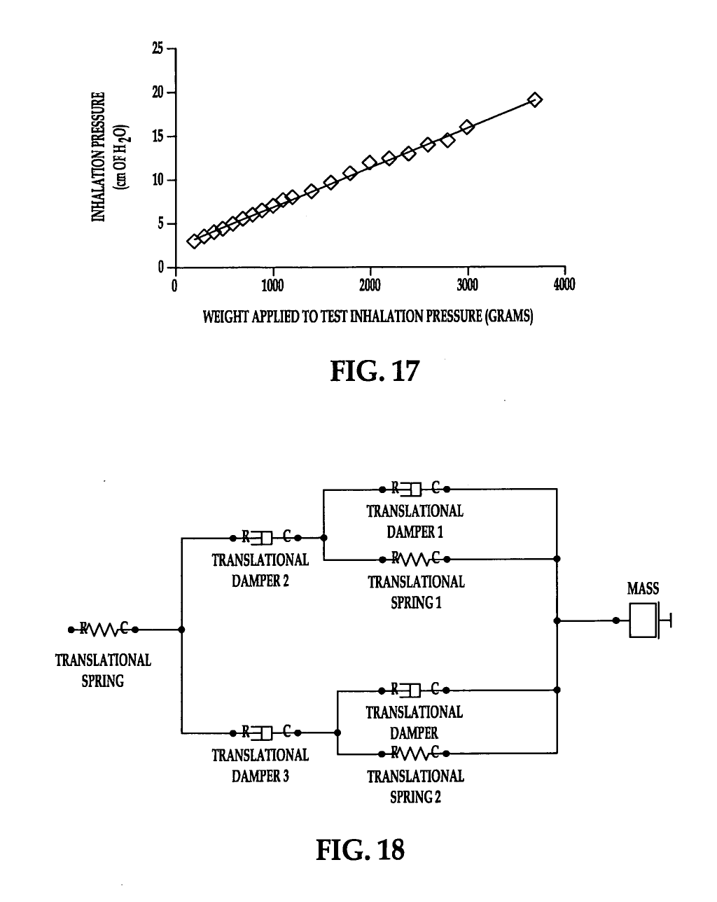

Referring to FIG. 16, the required exhalation pressure will vary among patients, but generally ranges between about 4 and 20 cm of H₂O. As shown by the graph, this pressure range corresponds to weights ranging between about 40 and 3,100 grams; this graph assumes that the first chamber has a volume of 650 ml and that the outer wall thereof has a diameter of 6¼ inches. The graph indicates a generally linear relationship between the amount of weight used and the pressure of exhaled breath required to move that weight. Referring now to FIG. 17, the pressure in which the fresh air is expelled at is determined in part by the amount of weight used. The figure indicates a generally linear relationship between the amount of weight used and the resulting pressure of fresh air expelled. Testing of various device designs and under different conditions may yield different results than shown in the graphs of FIGS. 16 and 17. Furthermore, it is believed that some energy could be lost in operation such as through friction which could also alter the results displayed in FIGS. 16 and 17. Using a prototype with the weight of a 500 gram mass, the first chamber required about 5 cm of H₂O pressure to inflate and resulted in about 4.5 cm of H₂O being expelled from the second chamber.

FIG. 18 shows a theoretical model of the sleep apnea treatment device 10 of the first embodiment as a simple spring, mass, and dampener system. The model was used to predict the motion of the movable plates. In FIG. 18, the symbol labeled mass represents the mass; the symbol labeled translational damper 1 represents friction generated during movement of the first outer wall of the first chamber; the symbol labeled translational spring 1 represents the elasticity provided by the first chamber; the symbol labeled translational damper represents friction generated during movement of the second outer wall of the second chamber; the symbol labeled translational spring 2 represents the elasticity provided by the second chamber; and the symbols labeled translational damper 2 and translational damper 3 represent friction generated by the rods during movement of the movable plates. This model gives an equation of motion for the system as:

m\ddot{x}+b_{eq}\dot{x}+k_{eq}x=F(t)

where b_{eq} is the equivalent combined damping coefficient of the system, k_{eq} is the equivalent spring coefficient of the system, and F(t) is the force provided by the user (exhalation) or the weight (inhalation).

The embodiment illustrated in FIGS. 1-11 is a two-chamber design used for exchanging exhaled breath for positively-pressurized fresh air. It should be appreciated that the chambers themselves need not be of equal dimensions and volumes. For example, one embodiment could include a fresh air chamber that has a larger diameter than the exhalation chamber. This would result in a somewhat reduced aspiratory pressure, compared to expiratory pressure, but allow a patient to inhale a larger volume than had just previously been exhaled. This could allow the machine to better accommodate variations that may occur in otherwise regular breathing during sleep. Moreover, in other embodiments, the device can be implemented in different ways to store energy received from the patient's exhaled breath, including designs that do not utilize two chambers and/or that do not involve using a mass lifted by the patient's breath. For example, the energy could be stored using an elastic bladder expanded by energy from the patient's exhalation of breath, or could be converted from mechanical pressure into an electrical or other form of energy that is then used to provide fresh air back to the patient at supra-atmospheric pressure. Also, for mechanical implementations, the energy need not be stored as potential energy, but could instead be stored as kinetic energy, such as by a spinning mass that is caused to spin by the patient's exhaled breath. Other such variations will become apparent to those skilled in the art.

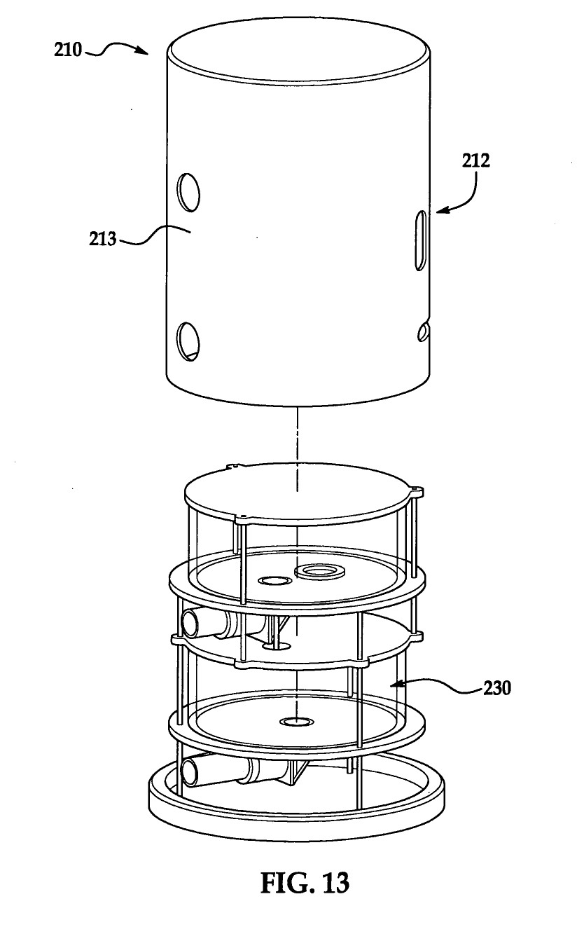

FIGS. 12 and 13 depict a second embodiment of a sleep apnea treatment device 210. The sleep apnea treatment device 210 is similar in some ways to that of the first embodiment, and similar components will not be described and repeated here. One difference of the device 210 is that the device has a cylindrically-shaped housing 212 with a one-piece outer shell 213. In this embodiment, an energy storage component 232 is a biasing member such as a compression spring 270. The compression spring 270 is interconnected between a moveable plate 236 and a stationary plate 274. In use, potential energy in the spring 270 increases as the spring is loaded. In other embodiments, the spring could be an expansion spring connected to one of the movable plates and to a stationary component, or could be a compression or expansion band.

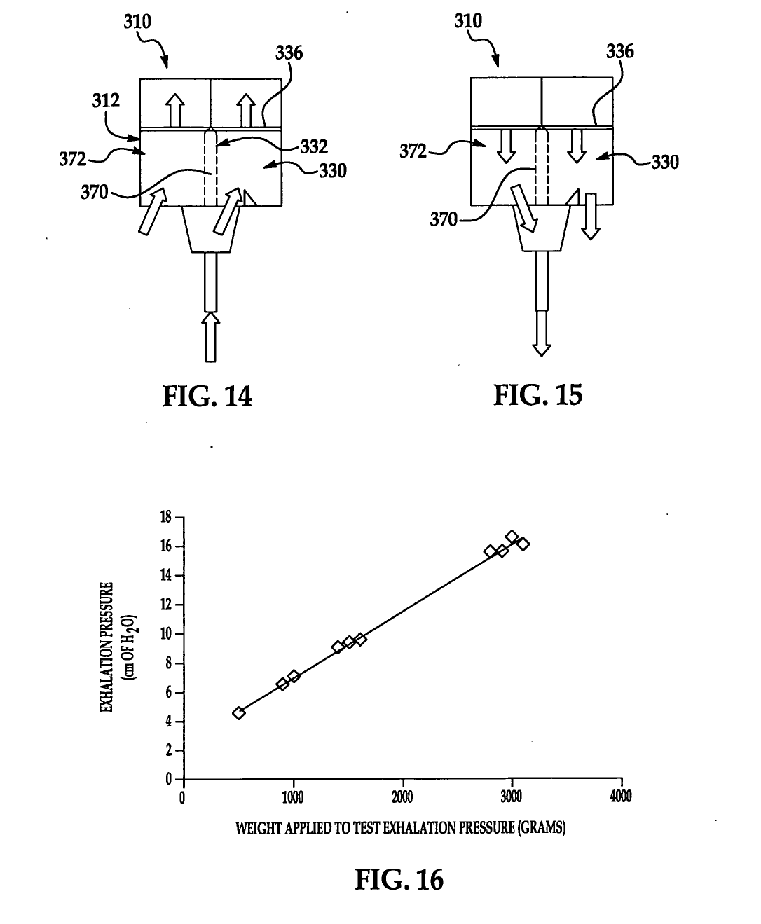

FIGS. 14 and 15 depict a schematically illustrated third embodiment of a sleep apnea treatment device 310. The sleep apnea treatment device 310 is similar in some ways to that of the first embodiment, and similar components will not be described and repeated here. One difference of the device 310 is that a first and second chamber 330, 372 share a common movable plate 336. And an expansion spring 370, which serves as an energy storage component 332, is interconnected between the movable plate 336 and a stationary housing 312. In other embodiments, the first and second chambers could be arranged in different ways such as being concentric with respect to each other where the first chamber is cylindrically-shaped, and the second chamber is donut-shaped surround the first chamber.

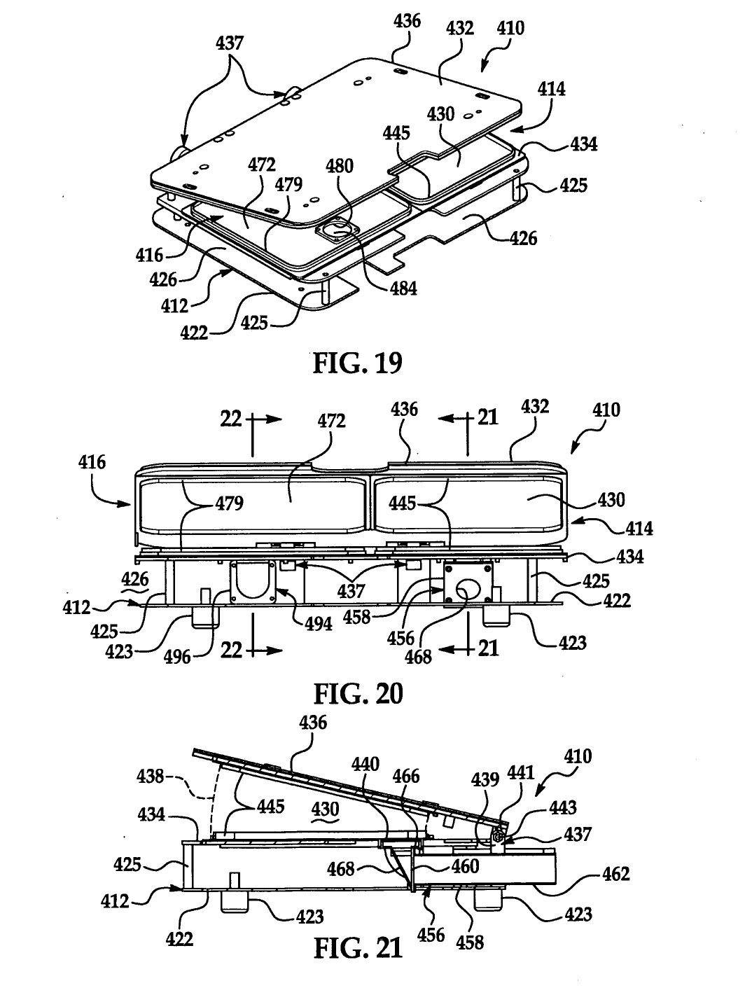

FIGS. 19-24 depict a fourth embodiment 410 of a sleep apnea treatment device. The sleep apnea treatment device 410 is in some ways similar to that of the first embodiment, and similar components may not necessarily be described and repeated here. Referring to FIGS. 19-22, a housing 412 includes a base 422 with four legs or stands 423 located near its corners to support the device 410 on an underlying surface. The base 422 is connected to, and vertically distanced from, a first plate 434 by way of four upright supports 425 located near corners of the base and plate. The housing 412 has open side walls 426, though one or more of the side walls could be closed partly or more by a side panel or screen.

An energy accumulator 414 in this embodiment includes a first chamber 430 and an energy storage component 432. The first, or exhalation, chamber 430 confines a first maximum volume which is the volume of gas it can contain when the first chamber is fully expanded. The first chamber 430 is defined in part by the first plate 434, a second plate 436, and an outer wall 438, which are joined together in an air-tight manner. The first and second plates 434, 436 have generally rectangular shapes. The first, or stationary, plate 434 does not move during use of the sleep apnea treatment device 410. It has a first opening or port 440 for the ingress and egress of exhaled breath in and out of the first chamber 430 during use of the device 410. The second, or movable, plate 436 is pivotally connected to the housing 412 by way of a pair of hinges 437, and during use of the device 410 moves up and down about a fulcrum defined at the hinges to expand and contract the size of the first chamber 430. Each hinge 437 includes a bracket 439 with a hole 441. The bracket 439 can be connected to the base 422 or to the first plate 434, and the hole 441 receives a rod 443 extending from the second plate 436. The rods 443 can be made of aluminum, steel, or another suitable rigid material such as a hard plastic, and could have a ground or polished outer surface to minimize friction with the contacting surfaces of the holes 441. The rods 443 turn in their respective holes 441 to define the fulcrum about which the second plate 436 pivots. In other embodiments, the second plate 436 can be hinged to the housing 412 or to the first plate 434 in different ways. For example, a single elongated rod can extend from the housing 412 or from the first plate 434 and can rotate in a sleeve extending from the second plate 436, or a pair of rods can extend from the housing or from the first plate and can respectively rotate in a pair of recesses in the second plate.

The outer wall 438 is shown in phantom in FIG. 21, and is not shown in FIGS. 19 and 20 so that other components of the sleep apnea treatment device 410 can be seen. The outer wall 438 extends and is interconnected between the first and second plates 434, 436, forms seals therewith, and allows the first chamber 430 to expand and contract in size. The outer wall 438 is made of a flexible material in the sense that it can be foldable, pliable, or otherwise capable of reciprocal collapsing and extending as breath is inhaled and exhaled, respectively. For example, the outer wall 438 can be made out of a bag material, a vinyl material, a PVDC material, can have a bellows-type construction, can be a flexible material supported in part by a skeletal wire, or can be another material and construction. In the figures, the outer wall 438 can be removed for periodic cleaning by the patient. The outer wall 438 and the plates 434, 436 are attached via a press- and snap-fit construction, where a male structure (not shown) is located at open ends of the outer wall, and complementary female structures 445 are located on surfaces of the plates. In other embodiments, the outer wall 438 can be attached to the plates 434, 436 in different ways. For example, the outer wall 438 can be attached to the first and second plates 434, 436 by adhesive, stitching, or stapling. In another embodiment, the first chamber 430 is defined primarily by a flexible wall like the outer wall 438 in the form of a bladder, for example, which is squeezed and stretched between the first and second plates 434, 436 for expansion and contraction.

Referring to FIGS. 20, 21, and 24, a first, or exhalation, valve assembly 456 communicates with the first chamber 430 and regulates ingress and egress of exhaled breath into and out of the first chamber. The first valve assembly 456 is located vertically between the base 422 and the first plate 434, and is attached to a bottom surface of the first plate for direct communication with the first opening 440. In the embodiment shown, the first valve assembly 456 includes a body 458 and a flapper 460. The body 458 can be made of a PVC material or another hard plastic or material, and can be manufactured by way of an injection molding process or another suitable process. The body 458 has an inlet tube 462 for connection to a hose assembly (not shown), an inlet port that is opened and closed by the flapper 460, a chamber port 466 that communicates directly with the first chamber 430, and an outlet port 468 that communicates directly with the inside of the housing's structure and indirectly with the atmosphere by way of the open side walls 426. The inlet tube 462 extends outside of the housing 412 beyond the open side wall 426 located at a backside of the housing

Referring particularly to FIG. 24, the flapper 460 is made of a rubber material and which can open one-way in a direction away from the inlet tube 462 and otherwise rests in a position where it closes the inlet port. In use, exhaled breath enters the valve assembly 456 through the inlet tube 462 and causes the flapper 460 to pivot exposing and opening the inlet port. When pivoted, the flapper 460 plugs the outlet port 468 so that the incoming exhaled breath does not exit the outlet port and instead passes through the chamber port 466 and into the first chamber 430. Conversely, when the first chamber 430 contracts the flapper 460 plugs the inlet port (i.e., it's at its resting position) and exiting exhaled breath passes through the chamber port 466 and through the outlet port 468. In other embodiments, the first valve assembly could be an off-the-shelf component purchased from a supplier and could have another construction.

Referring again to FIGS. 19-22, in this embodiment the mass of the second plate 436 serves as the energy storage component 432. The mass resists expansion of the first chamber 430 and upward pivotal movement of the second plate 436, and promotes contraction of the first chamber and downward pivotal movement of the second plate. With gravity, the mass exerts a constant weight or resistance force against upward pivotal movement of the second plate 436. In general, increasing potential energy is produced as the mass is displaced a vertical distance pivotally upward upon expansion of the first chamber 430. The mass can be provided by suitable selection of materials for the second plate 436 and thickness thereof. For example, the second plate 436 can be thicker at a location farther away from hinges 437 as compared to a location closer to the hinges. Locating a thickness and its associated mass farther away from the hinges 437 provides a greater resistance force than locating the mass closer to the hinges. In another embodiment, the energy storage component 432 can be provided, alone or in combination with the described mass, by an elongated rod of the hinges 437 which are pre-loaded and act as torsional bars that are biased in the downward pivotal direction and resist upward pivotal movement; and in yet another embodiment, the energy storage component can be provided, alone or in combination with the described mass, by a helical spring wound around one or more of the rods of the hinges and which is fixed at its ends to store mechanical energy upon upward pivotal movement of the second plate 436.

An air delivery subsystem 416 includes a second chamber 472 that draws-in fresh air during use of the sleep apnea treatment device 410. The second, or inhalation, chamber 472 confines a second maximum volume which has a greater value than the first maximum volume of the first chamber 430. In this way, the second chamber can draw-in a greater volume of fresh air than the volume of breath exhaled by the patient. In other embodiments, the first and second maximum volumes can have the same value. The second chamber 472 is defined in part by the first plate 434, the second plate 436, and an outer wall 478, which are joined together in an air-tight manner. The first and second chambers 430, 472 share the common first and second plates 434, 436, but are defined by different portions of the first and second plates and are located side-by-side with respect to each other. By both interacting with the first and second plates 434, 436, the first and second chambers 430, 472 function together and simultaneously expand and contract in size upon the single pivotal movement of the second plate 436.

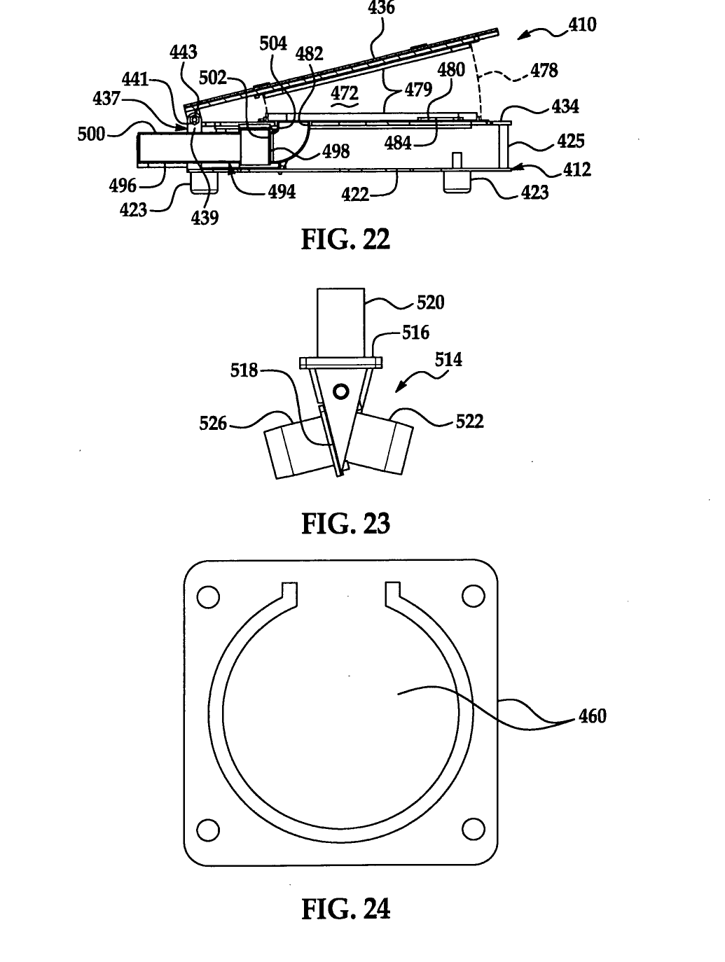

The outer wall 478 is shown in phantom in FIG. 22, and is not shown in FIGS. 19 and 20 so that other components of the sleep apnea treatment device 410 can be seen. The outer wall 478 extends and is interconnected between the first and second plates 434, 436, forms seals therewith, and allows the second chamber 472 to expand and contract in size. The outer wall 478 is made of a flexible material in the sense that it can be foldable, pliable, or otherwise capable of reciprocal collapsing and extending as breath is inhaled and exhaled, respectively. For example, the outer wall 478 can be made out of a bag material, a vinyl material, a PVDC material, can have a bellows-type construction, can be a flexible material supported in part by a skeletal wire, or can be another material and construction. In the figures, the outer wall 478 can be removed for periodic cleaning by the patient. The outer wall 478 and the plates 434, 436 are attached via a press- and snap-fit construction, where a male structure (not shown) is located at open ends of the outer wall, and complementary female structures 479 are located on surfaces of the plates. In other embodiments, the outer wall 478 can be attached to the plates 434, 436 in different ways. For example, the outer wall 478 can be attached to the first and second plates 434, 436 by adhesive, stitching, or stapling. In another embodiment, the second chamber 472 is defined primarily by a flexible wall like the outer wall 478 in the form of a bladder, for example, which is squeezed and stretched between the first and second plates 434, 436 for expansion and contraction. In yet another embodiment, the outer walls 438 and 478 can have a somewhat integral construction and can share a common dividing wall to separate the first and second volumes of the chambers 430, 472 from each other.

Referring to FIGS. 19 and 22, a second opening or port 480 is located in the first plate 434 for the ingress of fresh air from the atmosphere during use of the sleep apnea treatment device 410. A flapper 484, like that shown in FIG. 24, is fitted in the second opening 480 and is made of a rubber material. The flapper 484 rests in a closed position and opens one-way in a vertically upward direction to permit the entrance of fresh air into the second chamber 472 upon expansion of the second chamber the suction of expansion causing the flapper to open. When the second chamber 472 is being contracted, the flapper 484 closes the second opening 480 so that fresh air does not exit the second chamber by way of the second opening.

Referring to FIGS. 20, 22, and 24, a second, or inhalation, valve assembly 494 communicates with the second chamber 472 and regulates the egress of fresh air out of the second chamber. The second valve assembly 494 is located vertically between the base 422 and the first plate 434, and is attached to a bottom surface of the first plate for direct communication with a third opening or port 482. In this embodiment, the second valve assembly 494 includes a body 496 and a flapper 498 like the flapper shown in FIG. 24. The body 496 can be made of a PVC material or another hard plastic or material, and can be manufactured by way of an injection molding process, or another suitable process. The body 496 has an outlet tube 500 for connection to the hose assembly, an inlet port 502 that is opened and closed by the flapper 498, and a chamber port 504 that communicates directly with the second chamber 472 and with the third opening 482. Upon contraction of the second chamber 472, expelled fresh air enters the second valve assembly 494 through the chamber port 504 and causes the flapper 498 to pivot exposing and opening the inlet port 502. The expelled fresh air then travels through the outlet tube 500 and to the hose assembly. In other embodiments, the second valve assembly could be an off-the-shelf component purchased from a supplier and could have another construction.

Referring to FIG. 23, the hose assembly includes a valve assembly 514. Though not shown, the valve assembly 514 can be located at the intersection of multiple hoses of the hose assembly. The valve assembly 514 regulates gas flow from the patient to the first chamber 430 and from the second chamber 472 to the patient. The valve assembly 514 permits the patient's exhaled breath to flow from a first hose communicating with a patient's mask and to a second hose communicating with the first chamber 430, and permits the expelled and positively pressurized fresh air to flow from a third hose communicating with the second chamber 472 and to the first hose, prevents the patient's exhaled breath from flowing from the first hose and to the third hose, and prevents the fresh air from flowing from the third hose and to the second hose.

In this embodiment, the valve assembly 514 includes a body 516 and a flapper 518 like the flapper shown in FIG. 24. The body 516 can be made of a PVC material or another hard plastic or material, and can be manufactured by way of an injection molding process, or another suitable process. The body 516 has a first port 520 that communicates with the first hose, a second port 522 that communicates with the second hose, and a third port 524 that communicates with the third hose. The flapper 518 is made of a rubber material and opens one-way in the direction of the second port 522, and otherwise rests in a position where it plugs and closes the third port 524. In use, fresh air flowing from the third hose causes the flapper 518 to pivot and open the third port 524. When pivoted, the flapper 518 plugs and closes the second port 522. In other embodiments, the valve assembly could be an off-the-shelf component purchased from a supplier and could have another construction.

Note that in this embodiment, the second (inhalation) chamber 472 is larger than the first (exhalation) chamber 430. The inhalation chamber 472 is larger so that if air leaks from the system, or the apnea patient takes a suddenly larger breath for some reason, he or she will still have sufficient pressurized fresh air for the entire inhalation.

It is to be understood that the foregoing description is of one or more preferred exemplary embodiments of the invention. The invention is not limited to the particular embodiment(s) disclosed herein, but rather is defined solely by the claims below. Furthermore, the statements contained in the foregoing description relate to particular embodiments and are not to be construed as limitations on the scope of the invention or on the definition of terms used in the claims, except where a term or phrase is expressly defined above. Various other embodiments and various changes and modifications to the disclosed embodiment(s) will become apparent to those skilled in the art. For example, in some embodiments a separate power source can be utilized, such as to provide supplemental energy in generating the pressurized volume of fresh air, or to provide device monitoring or data gathering relating to the functioning of the device and/or patient. Also, as discussed above, rather than lifting a mass against the force of gravity to store energy from the patient's expiratory breath, in another embodiments other energy storage approaches can be used; for example, using a mass that slides horizontally perhaps against a spring force to store the energy. All such other embodiments, changes, and modifications are intended to come within the scope of the appended claims.

As used in this specification and claims, the terms “for example,” “for instance,” and “such as,” and the verbs “comprising,” “having,” “including,” and their other verb forms, when used in conjunction with a listing of one or more components or other items, are each to be construed as open-ended, meaning that the listing is not to be considered as excluding other, additional components or items. Other terms are to be construed using their broadest reasonable meaning unless they are used in a context that requires a different interpretation.

1. A positive airway pressure device, comprising
an energy accumulator including a first port that receives exhaled breath from a patient and at least one component that stores energy from the received breath; and
an air delivery subsystem having a second port and being coupled to the energy accumulator, wherein the air delivery subsystem generates a pressurized volume of fresh air using the stored energy and provides the pressurized volume of fresh air to the second port for delivery to the patient.

2. A sleep apnea treatment device, comprising:
a first chamber receiving exhaled breath from a patient and being inflated by the exhaled breath;
a second chamber expanding in size in response to the inflation of the first chamber and drawing in fresh air upon expansion to be inhaled by the patient; and
an energy storage component interacting with the second chamber;
wherein, upon termination of the patient's exhaled breath and initiation of inhalation, the energy storage component facilitates contraction of the second chamber and the drawn-in fresh air is expelled under positive pressure out of the second chamber for delivery to the patient.

3. A sleep apnea treatment device as defined in claim 2, wherein the device is a passive system in which the second chamber is expanded and contracted in size without the assistance of an external power source.

4. A sleep apnea treatment device as defined in claim 2, wherein the first chamber is defined at least in part by a flexible wall, and the second chamber is defined at least in part by a flexible wall.

5. A sleep apnea treatment device as defined in claim 4, wherein the first chamber is further defined at least in part by a first stationary plate and a first movable plate, and the second chamber is further defined at least in part by a second stationary plate and a second movable plate, wherein the first and second movable plates are connected to each other and move in unison with each other to expand and contract the size of the first and second chambers.

6. A sleep apnea treatment device as defined in claim 4, wherein the first chamber is further defined at least in part by a stationary plate and a movable plate, and the second chamber is further defined at least in part by the stationary plate and the movable plate, wherein the movable plate moves to simultaneously expand and contract the size of the first and second chambers.

7. A sleep apnea treatment device as defined in claim 2, wherein the energy storage component comprises a mass which is moved by expansion and contraction of the second chamber and which exerts a weight to the second chamber to bias the second chamber to a contracted state.

8. A sleep apnea treatment device as defined in claim 2, wherein the energy storage component comprises a spring which is loaded upon expansion of the second chamber and which exerts a spring force to facilitate contraction of the second chamber.

9. A sleep apnea treatment device as defined in claim 2, further comprising:
a first valve assembly communicating with the first chamber and directing flow of incoming exhaled breath into the first chamber through a port, and directing flow of outgoing exhaled breath out of the first chamber upon contraction of the first chamber; and
a second valve assembly communicating with the second chamber and closing a port of the second chamber upon expansion of the second chamber, and opening the port upon contraction of the second chamber to direct expelled fresh air out of the second chamber.

10. A sleep apnea treatment device as defined in claim 2, further comprising:
at least a pair of plates and a flexible wall defining at least a part of the first chamber, the second chamber, or both of the first and second chambers;
a housing connected to at least one of the pair of plates; and
a hose assembly connected to the first and second chambers, and communicating the patient's exhaled breath to the first chamber and communicating the positively pressurized fresh air out of the second chamber to the patient.

11. A sleep apnea treatment device, comprising:
a housing;
a first expandable and contractible chamber defined at least in part by a stationary plate connected to the housing, a movable plate, and a first outer wall connected between the stationary plate and the movable plate;
a second expandable and contractible chamber defined at least in part by the stationary plate, the movable plate, and a second outer wall connected between the stationary plate and the movable plate; and
an energy storage component interacting with the movable plate to bias the movable plate to a position in which the first and second chambers are contracted in size;
wherein, upon exhalation of a patient, the first chamber receives the exhaled breath and is inflated by the exhaled breath and expands in size, the movable plate moves and causes the second chamber to expand in size and the second chamber draws-in fresh air upon expansion, and wherein, upon inhalation of the patient, the energy storage component facilitates movement of the movable plate to contract the size of the first and second chambers whereupon the exhaled breath leaves the first chamber to the atmosphere and the fresh air is expelled under positive pressure out of the second chamber for delivery to the patient.

12. A sleep apnea treatment device as defined in claim 11, wherein the first chamber expands to a maximum first volume and the second chamber expands to a maximum second volume which is greater than the maximum first volume.

13. A sleep apnea treatment device as defined in claim 11, wherein the movable plate is hinged and pivots about its hinge when moved to expand and contract the size of the first and second chambers.

14. A sleep apnea treatment device as defined in claim 11, wherein the mass of the movable plate serves as the energy storage component and exerts a weight which biases the movable plate to the position in which the first and second chambers are contracted in size.

15. A sleep apnea treatment device as defined in claim 11, further comprising:
a first valve assembly communicating with the first chamber and directing flow of incoming exhaled breath into the first chamber through a port, and directing flow of outgoing exhaled breath out of the first chamber upon contraction of the first chamber; and
a second valve assembly communicating with the second chamber and closing a port of the second chamber upon expansion of the second chamber, and opening the port upon contraction of the second chamber to direct expelled fresh air out of the second chamber.

16. A sleep apnea treatment device as defined in claim 11, further comprising a hose assembly connected to the first and second chambers and communicating the patient's exhaled breath to the first chamber and communicating the positively pressurized fresh air out of the second chamber to the patient, the hose assembly comprising a valve assembly with a first port communicating exhaled breath from the patient and communicating fresh air to the patient, a second port communicating exhaled breath to the first chamber, and a third port communicating fresh air from the second chamber, the valve assembly including a flapper that closes the third port when the patient is exhaling and that closes the second port when the patient is inhaling.

17. A method of treating sleep apnea, comprising the steps of:
storing energy received from exhalation of air by a patient;
creating a pressurized volume of fresh air using the stored energy; and
delivering the fresh air to the patient during inspiration.

18. The method of claim 17, wherein the storing step further comprising storing the energy as potential energy.
